(19) United States
(12) Patent Application Publication
(10) Pub. No.: US 2026/0263180 A1
(43) Pub. Date: Sep. 10, 2026

(54) INTRAOPERATIVE EXCESSIVE BODY WALL FORCE DETECTION AND MITIGATION

(71) Applicant: Auris Health, Inc., Santa Clara, CA (US)

(72) Inventors: Avinash SIRAVURU, Santa Clara, CA (US); Shaobo WANG, Santa Clara, CA (US); Jinxin ZHAO, Santa Clara, CA (US)

(21) Appl. No.: 19/661,595

(22) Filed: Apr. 28, 2026

Related U.S. Application Data
(63) Continuation of application No. PCT/IB2024/060985, filed on Nov. 6, 2024.
(60) Provisional application No. 63/596,599, filed on Nov. 6, 2023.

(51) Int. Cl. A61B 34/37; A61B 34/20; A61B 50/13; A61B 90/00; A61G 13/02; G16H 40/67

(52) U.S. Cl. CPC A61B 34/37; A61B 50/13; A61G 13/02; G16H 40/67; A61B 2034/2059; A61B 2090/031; A61B 2090/064

(57) ABSTRACT

Disclosed are systems and methods for detecting and mitigating intraoperative excessive body wall force. A compliance mode may be enabled for controlling a robotic arm of a surgical robot to allow a remote center of motion (RCM) of the robotic arm to be compliant with an external force. An RCM displacement may be determined under the compliance mode in response to the external force, and responsive to determining that the RCM displacement exceeds a maximum threshold, disabling the compliance mode and sending a notification.

### CROSS-REFERENCE

This Application is a continuation of International Application No. PCT/IB2024/060985, filed Nov. 6, 2024, which claims the benefit of the U.S. Provisional Patent Application No. 63/596,599, filed Nov. 6, 2023, each of which is herein incorporated by reference in its entirety.

### TECHNICAL FIELD

This disclosure generally relates to surgical robotic systems, and more specifically to robotics and control of a surgical robotic system for minimally invasive surgeries.

### BACKGROUND

Minimally invasive surgery (MIS), such as laparoscopic surgery, involves techniques intended to reduce tissue damage during a surgical procedure. For example, laparoscopic procedures typically involve creating several small incisions in the patient (e.g., in the abdomen), and introducing one or more surgical tools (e.g., end effectors and endoscope) through the incisions into the patient. The surgical procedures may then be performed using the introduced surgical tools, with the visualization aid provided by the endoscope.

Generally, MIS provides multiple benefits, such as reduced patient scarring, less patient pain, shorter patient recovery periods, and lower medical treatment costs associated with patient recovery. Recent technology development allows more MIS to be performed with robotic systems that include one or more robotic arms for manipulating surgical tools based on commands from a remote operator. A robotic arm may, for example, support at its distal end various devices such as surgical end effectors, imaging devices, cannulas for providing access to the patient's body cavity and organs, etc. In robotic MIS systems, it may be desirable to establish and maintain high positional accuracy for surgical instruments supported by the robotic arms.

Surgeons may also need to re-orient the surgical table and patient quickly and safely during robot-assisted teleoperation. Current surgical robotic systems have a de-coupled design, in which robotic manipulators are mounted on a different platform than the patient on a surgical table. Making intraoperative adjustment of the table while maintaining the port placements on the patient becomes very challenging, if not impossible. Moreover, the surgical table model may need to be known apriori by the robotic system to ensure the safety of the procedure. At the expense of additional downtime time, some systems may take a two-step approach: (1) allow the port positions to be changed during table adjustment, and then (2) prompt bed-side staff to re-adjust trocars to their intended locations. It is desirable to have mechanical and software enablement for easy and safe intraoperative table adjustment or intraoperative arm-mounted cart adjustment, while maintaining trocar positions and not causing additional harm to patients.

### SUMMARY

Disclosed herein is a robotic assisted surgical system, which is a software-controlled, electro-mechanical system, designed for surgeons to perform minimally invasive surgery.

In some implementations, the robotic assisted surgical system comprises a control process. The control process includes enabling a compliance mode in controlling a robotic arm of a surgical robot, wherein the robotic arm includes a plurality of joints and a remote center of motion (RCM) aligned with an incision port on body wall of a patient, and the compliance mode allows the RCM to be compliant with an external force exerted by the body wall around the incision port. The control process next determines an RCM displacement under the compliance mode in response to the external force and estimating the external force based the determined RCM displacement. Next, responsive to determining the external force exceeding a safety force threshold or a predetermined max displacement reached, the control process also includes disabling the compliance mode and sending a notification.

The control process further comprises enabling the compliance mode by determining a desired RCM stiffness based on safety requirements including the safety force threshold and/or the predetermined max displacement of the RCM; applying a kinematic model of the robotic arm to compute a Jacobian matrix between the RCM and the plurality of joints; mapping the desired RCM stiffness to corresponding joint stiffness at each of the plurality of joints based on the Jacobian matrix; and applying a spring mass damper model at each of the plurality of joints based on the mapped joint stiffness. Wherein the external force is determined based on the desired RCM stiffness and the determined RCM displacement, and wherein the external force is substantially proportional to the RCM displacement by a factor of the desired RCM stiffness. In addition, the spring mass damper model is applied using admittance control that generates a position output based on torque input at each of the plurality of joints. Alternatively, the spring mass damper model is applied using impedance control that generates a joint torque output based on current joint angle and desired joint angle at each of the plurality of joints. Included in the control process, the RCM displacement is determined based on displacements sensed at the plurality of joints. Furthermore, the external force on the RCM is caused by a patient re-orientation relative to the RCM, wherein the patient re-orientation is caused by motions of a surgical table, on which the patient is disposed, wherein the notification to the user of the surgical robot comprises a warning to stop all table motions. The external force on the RCM can also be caused by RCM displacement.

According to one embodiment of the disclosure, a method includes: enabling a compliance mode in controlling a robotic arm of a surgical robot to allow a remote center of motion (RCM) of the robotic arm to be compliant with an external force exerted; determining an RCM displacement under the compliance mode in response to the external force; and responsive to determining that the RCM displacement exceeds a maximum threshold, disabling the compliance mode and sending a notification.

In one embodiment, the robotic arm is mounted on a surgical table on which a patient is disposed, the method further includes causing movement of the surgical table, the external force is exerted on the RCM by an incision port on body wall of the patient as the movement of the surgical table occurs. In another embodiment, the method further includes pausing the movement of the surgical table responsive to the determining that the RCM displacement exceeds the maximum threshold or to receiving a user command after the notification is sent.

In one embodiment, the robotic arm includes a group of joints, enabling the compliance mode includes: determining a desired RCM stiffness; applying a kinematic model of the robotic arm to compute a Jacobian matrix between the RCM and the group of joints; mapping the desired RCM stiffness to corresponding joint stiffness at each of the group of joints based on the Jacobian matrix; and applying a spring mass damper model at each of the group of joints based on the mapped joint stiffness. In another embodiment, the method further includes estimating the external force based on the desired RCM stiffness and the determined RCM displacement, the compliance mode is disabled, and the notification is sent responsive to determining that the external force exceeds a force threshold. In some embodiments, the external force is proportional to the RCM displacement by a factor of the desired RCM stiffness. In another embodiment, the spring mass damper model is applied using either 1) an admittance control that generates a position output based on torque input at each of the group of joints or 2) impedance control that generates a joint torque output based on current joint angle and desired joint angle at each of the group of joints.

In one embodiment, a patient is disposed on a surgical table and the external force is caused by a portion of the patient pushing up against a surgical instrument coupled to the robotic arm due to surgical table motion, the notification includes a warning for a user of the surgical robot to stop all surgical table motions.

According to another embodiment of the disclosure includes a surgical robotic system, that includes: a robotic arm having a group of joints and a remote center of motion (RCM) aligned with an incision port on a body wall of a patient; at least one processor; and memory having instructions stored therein which when executed by the at least one processor causes the surgical robotic system to: enable a compliance mode in controlling the robotic arm, the compliance mode allows the RCM to be compliant with an external force exerted by the body wall around the incision port, determine an RCM displacement under the compliance mode in response to the external force; and responsive to determining that the RCM displacement has reached a maximum displacement of the RCM, disable the compliance mode and send a notification.

In one embodiment, the memory has further instructions to estimate the external force exerted by the body wall based on the determined RCM displacement, the compliance mode is disabled, and the notification is sent responsive to determining that the external force exceeds a force threshold, and that the RCM displacement has reached the determined maximum displacement.

In one embodiment, the instructions to enable the compliance mode includes instructions to: determine a desired RCM stiffness based on the force threshold and the maximum displacement of the RCM; apply a kinematic model of the robotic arm to compute a Jacobian matrix between the RCM and the group of joints; map the desired RCM stiffness to corresponding joint stiffness at each of the group of joints based on the Jacobian matrix; and apply a spring mass damper model at each of the group of joints based on the mapped joint stiffness. In another embodiment, the external force is determined based on the desired RCM stiffness and the determined RCM displacement, the external force is proportional to the RCM displacement by a factor of the desired RCM stiffness. In some embodiments, the spring mass damper model is applied using either 1) an admittance control that generates a position output based on torque input at each of the group of joints or 2) impedance control that generates a joint torque output based on current joint angle and desired joint angle at each of the group of joints.

In one embodiment, the patient is disposed on a surgical table and the external force is caused by surgical table motion, wherein the notification includes a warning for a user to stop all surgical table motion. In another embodiment, the robotic arm is mounted on the surgical table. In some embodiments, the robotic arm is mounted on a cart that is separate from the surgical table.

According to another embodiment of the disclosure, a non-transitory machine-readable medium storing instructions operable to cause one or more processors to perform operations including: enabling a compliance mode in controlling a robotic arm of a surgical robot, wherein the robotic arm includes a remote center of motion (RCM) aligned with an incision port on a body wall of a patient, and the compliance mode allows the RCM to be compliant with an external force exerted by the body wall around the incision port; determining an RCM displacement under the compliance mode in response to the external force; estimating the external force based on the determined RCM displacement; and responsive to determining that the external force exceeds a force threshold or the RCM displacement reaches a maximum displacement, disabling the compliance mode and sending a notification.

In one embodiment, the robotic arm includes a group of joints, enabling the compliance mode includes: determining a desired RCM stiffness based on the force threshold or the maximum displacement; applying a kinematic model of the robotic arm to compute a Jacobian matrix between the RCM and the group of joints; mapping the desired RCM stiffness to corresponding joint stiffness at each of the group of joints based on the Jacobian matrix; and applying a spring mass damper model at each of the group of joints based on the mapped joint stiffness. In another embodiment, the external force is determined based on the desired RCM stiffness and the determined RCM displacement, the external force is proportional to the RCM displacement by a factor of the desired RCM stiffness.

In one embodiment, the external force on the RCM is caused by a patient re-orientation relative to the RCM. In another embodiment, the patient re-orientation is caused by motions of a surgical table, on which the patient is disposed.

The above summary does not include an exhaustive list of all embodiments of the disclosure. It is contemplated that the disclosure includes all systems and methods that can be practiced from all suitable combinations of the various embodiments summarized above, as well as those disclosed in the Detailed Description below and particularly pointed out in the claims. Such combinations may have particular advantages not specifically recited in the above summary.

### BRIEF DESCRIPTION OF THE DRAWINGS

The embodiments are illustrated by way of example and not by way of limitation in the figures of the accompanying drawings in which like references indicate similar elements. It should be noted that references to “an” or “one” embodiment of this disclosure are not necessarily to the same embodiment, and they mean at least one. Also, in the interest of conciseness and reducing the total number of figures, a given figure may be used to illustrate the features of more than one embodiment, and not all elements in the figure may be required for a given embodiment.

FIG. 1 is a diagram illustrating an example operating room environment with a surgical robotic system, in accordance with aspects of the subject technology.

FIG. 2 is a schematic diagram illustrating one exemplary design of a robotic arm, a tool drive, and a cannula loaded with a robotic surgical tool, in accordance with aspects of the subject technology.

FIG. 3 is a flowchart illustrating a workflow for detecting and mitigating excessive body wall force intraoperatively, in accordance with aspects of the subject technology.

FIG. 4 is a is a flowchart illustrating an example design of compliance at remote center of motion (RCM) for detecting and mitigating excessive body wall force intraoperatively, in accordance with aspects of the subject technology.

FIG. 5 is a block diagram illustrating a joint that includes one example design of a joint-level controller for RCM compliance, in accordance with aspects of the subject technology.

FIG. 6 is a block diagram illustrating a joint that includes another example design of a joint-level controller for RCM compliance, in accordance with aspects of the subject technology.

### DETAILED DESCRIPTION

Several embodiments of the disclosure with reference to the appended drawings are now explained. Whenever the shapes, relative positions and other embodiments of the parts described in a given embodiment are not explicitly defined, the scope of the disclosure here is not limited only to the parts shown, which are meant merely for the purpose of illustration. Also, while numerous details are set forth, it is understood that some embodiments may be practiced without these details. In other instances, well-known circuits, structures, and techniques have not been shown in detail so as not to obscure the understanding of this description. Furthermore, unless the meaning is clearly to the contrary, all ranges set forth herein are deemed to be inclusive of each range's endpoints.

### System Overview

A robotic-assisted surgical system disclosed herein is a software-controlled, electro-mechanical system designed for surgeons to perform minimally invasive surgery. The surgical robotic system can be used with an endoscope, compatible endoscopic instruments, and accessories. The system may be used by trained physicians in an operating room environment to assist in the accurate control of compatible endoscopic instruments during robotically assisted urologic, gynecologic, and other laparoscopic surgical procedures. The system also allows the surgical staff to reposition the patient by adjusting the table without undocking the robotic arms during urologic, gynecologic, and other laparoscopic surgical procedures. The compatible endoscopic instruments and accessories for use with the surgical system are intended for endoscopic manipulation of tissue including grasping, cutting, blunt and sharp dissection, approximation, ligation, electrocautery, and suturing.

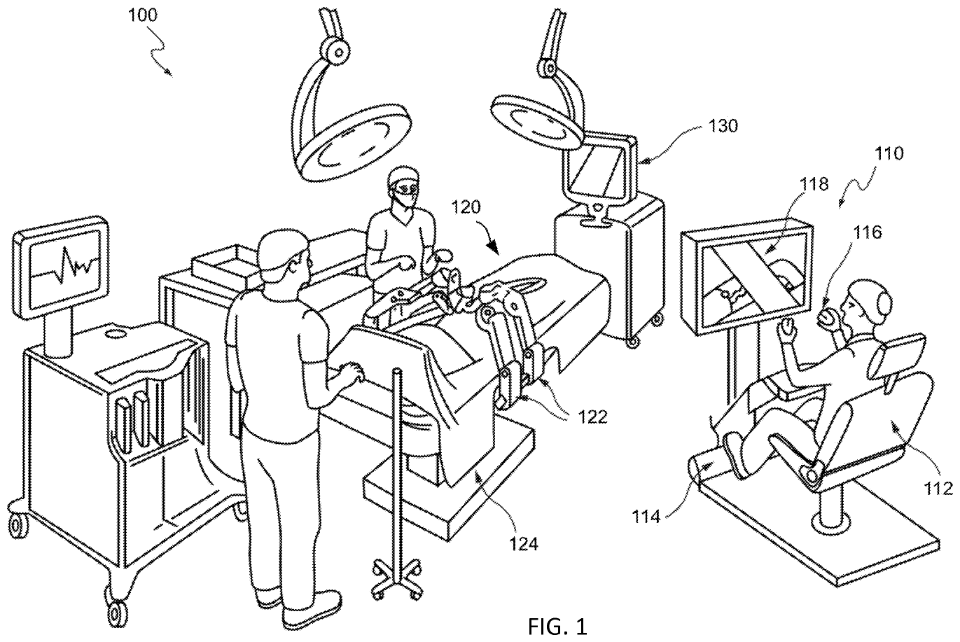

FIG. 1 is a diagram illustrating an example operating room environment with a surgical robotic system 100, in accordance with aspects of the subject technology. As shown in FIG. 1, the surgical robotic system 100 comprises a user console 110, a control tower 130, and a surgical robot 120 having one or more surgical robotic arms 122 mounted on a surgical platform 124 (e.g., a table or a bed etc.), where surgical tools with end effectors are attached to the distal ends of the robotic arms 122 for executing a surgical procedure. The robotic arms 122 are shown as table-mounted, but in other configurations, the robotic arms may be mounted in a cart, a ceiling, a sidewall, or other suitable support surfaces.

Generally, a user, such as a surgeon or other operator, may be seated at the user console 110 to remotely manipulate the robotic arms 122 and/or surgical instruments (e.g., teleoperation). The user console 110 may be in the same operation room as the robotic system 100, as shown in FIG. 1. In other environments, the user console 110 may be in an adjacent or nearby room, or tele-operated from a remote location in a different building, city, or country. The user console 110 may comprise a seat 112, pedals 114, one or more handheld human interface devices (HIDs) 116, and an open display 118 configured to display, for example, a view of the surgical site inside a patient. As shown in the exemplary user console 110, a surgeon siting in the seat 112 and viewing the open display 118 may manipulate the pedals 114 and/or handheld human interface devices 116 to remotely control the robotic arms 122 and/or surgical instruments mounted to the distal ends of the arms.

In some variations, a user may also operate the surgical robotic system 100 in an “over the bed” (OTB) mode, in which the user is at the patient's side and simultaneously manipulating a robotically driven tool/end effector attached thereto (e.g., with a handheld human interface device 126 held in one hand) and a manual laparoscopic tool. For example, the user's left hand may be manipulating a handheld human interface device 116 to control a robotic surgical component, while the user's right hand may be manipulating a manual laparoscopic tool. Thus, in these variations, the user may perform both robotic-assisted MIS and manual laparoscopic surgery on a patient.

During an exemplary procedure or surgery, the patient is prepped and draped in a sterile fashion to achieve anesthesia. Initial access to the surgical site may be performed manually with the robotic system 100 in a stowed configuration or withdrawn configuration to facilitate access to the surgical site. Once the access is completed, initial positioning and/or preparation of the robotic system may be performed. During the procedure, a surgeon in the user console 110 may utilize the pedals 114 and/or human interface devices 116 to manipulate various end effectors and/or imaging systems to perform the surgery. Manual assistance may also be provided at the procedure table by sterile-gowned personnel, who may perform tasks including but not limited to, retracting tissues, or performing manual repositioning or tool exchange involving one or more robotic arms 122. Non-sterile personnel may also be present to assist the surgeon at user console 110. When the procedure or surgery is completed, the robotic system 100 and/or user console 110 may be configured or set in a state to facilitate one or more post-operative procedures, including but not limited to, robotic system 100 cleaning and/or sterilization, and/or healthcare record entry or printout, whether electronic or hard copy, such as via the user console 110.

In some aspects, the communication between the surgical robot 120 and the user console 110 may be through the control tower 130, which may translate user input from the user console 110 to robotic control commands and transmit the control commands to the surgical robot 120. The control tower 130 may also transmit status and feedback from the robot 120 back to the user console 110. The connections between the surgical robot 120, the user console 110 and the control tower 130 may be via wired and/or wireless connections and may be proprietary and/or performed using any of a variety of data communication protocols. Any wired connections may be optionally built into the floor and/or walls or ceiling of the operating room. The surgical robotic system 100 may provide video output to one or more displays, including displays within the operating room as well as remote displays accessible via the Internet or other networks. The video output or feed may also be encrypted to ensure privacy and all or portions of the video output may be saved to a server or electronic healthcare record system.

Prior to initiating surgery with the surgical robotic system, the surgical team can perform the preoperative setup. During the preoperative setup, the main components of the surgical robotic system (table and robotic arms, control tower and user console) are positioned in the operating room, connected, and powered on. The table and robotic arms may be in a fully stowed configuration with the arms under the table. The surgical team can extend the arms from their stowed position for sterile draping. After draping, the arms can be partially retracted until needed for use. A number of conventional laparoscopic steps may need to be performed including trocar placement and insufflation. For example, each sleeve can be inserted with the aid of an obturator, into a small incision and through the body wall. The sleeve and obturator allow optical entry for visualization of tissue layers during insertion to minimize risk of injury during placement. The endoscope is typically placed first to provide handheld camera visualization for placement of other trocars. After insufflation, if required, manual instruments can be inserted through the sleeve to perform any laparoscopic steps by hand.

Next, the surgical team may position the robotic arms over the patient and attach each arm to its corresponding sleeve. The surgical robotic system has the capability to uniquely identify each tool (endoscope and surgical instruments) as soon as it is attached and display the tool type and arm location on the open or immersive display at the user console and the touchscreen display on the control tower. The corresponding tool functions are enabled and can be activated using the master HIDs and foot pedals. The patient side assistant can attach and detach the tools, as required, throughout the procedure. The surgeon seated at the user console can begin to perform surgery using the tools controlled by two master HIDs and foot pedals. The system translates the surgeon's hand, wrist, and finger movements through the master HIDs into precise real-time movements of the surgical tools. Therefore, the system constantly monitors every surgical maneuver of the surgeon and pauses instrument movement if the system is unable to precisely mirror the surgeon's hand motions. In case the endoscope is moved from one arm to another during surgery, the system can adjust the master HIDs for instrument alignment and continue instrument control and motion. The foot pedals may be used to activate various system modes, such as endoscope control and various instrument functions including monopolar and bipolar cautery, without involving surgeon's hands removed from the master HIDs.

The table can be repositioned intraoperatively. For safety reasons, all tool tips should be in view and under active control by the surgeon at the user console. Instruments that are not under active surgeon control must be removed and the table feet must be locked. During table motion the integrated robotic arms may passively follow the table movements. Audio and visual cues can be used to guide the surgery team during table motion. Audio cues may include tones and voice prompts. Visual messaging on the displays at the user console and control tower can inform the surgical team of the table motion status.

### Robotic Arms and Table

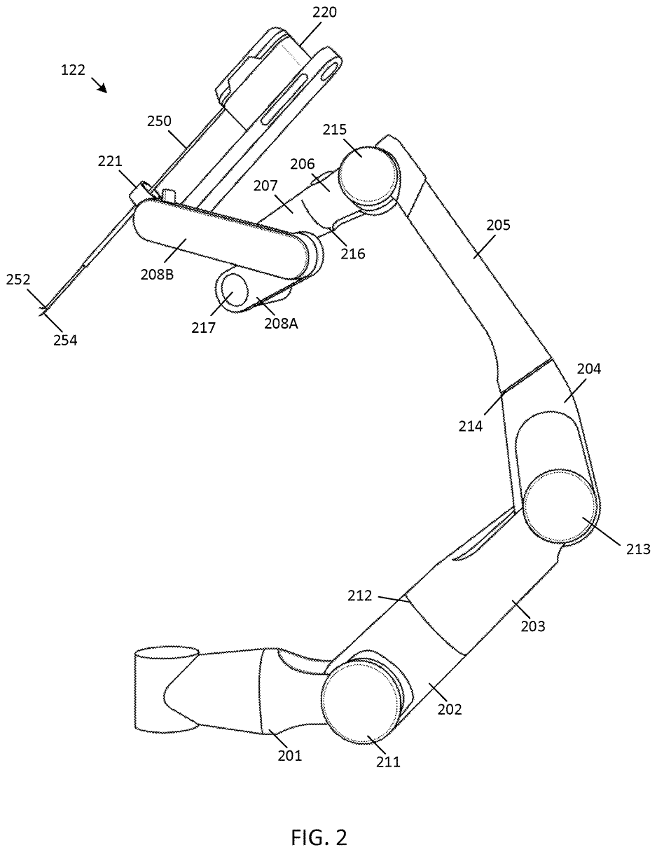

FIG. 2 is a schematic diagram illustrating one exemplary design of a robotic arm, a tool drive, and a cannula loaded with a robotic surgical tool, in accordance with aspects of the subject technology. As shown in FIG. 2, the example surgical robotic arm 122 may include a plurality of links (e.g., links 201-208) and a plurality of actuated joint modules (e.g., joints 211-217) for actuating the plurality of links relative to one another. The joint modules may include various types, such as a pitch joint or a roll joint, which may substantially constrain the movement of the adjacent links around certain axes relative to others. Also shown in the exemplary design of FIG. 2 is a tool drive 220 attached to the distal end of robotic arm 122. The tool drive 220 may include a cannula 221 coupled to its end to receive and guide a surgical instrument 250 (e.g., endoscopes, staplers, etc.). The surgical instrument (or “tool”) 250 may include an end effector having a robotic wrist 252 and an end effector (e.g., jaws) 254 at the distal end of the tool. The plurality of the joint modules of robotic arm 122 can be actuated to position and orient the tool drive 220, which actuates the robotic wrist 252 and the end effector 254 for robotic surgeries.

In some variations, the plurality of links and joints of the robotic arm can be divided into two segments. The first segment (setup or Cartesian arm) includes links 201-205 and joints 211-215 that provide at least five degrees of freedom (DOFs). The proximal end of the first segment can be mounted to a fixture, while the distal end is coupled to the second segment. The second segment (spherical arm) includes links 206-208 providing the arm with at least two DOFs. Link 208 may comprise a first link 208A and a second link 208B operatively coupled with a pulley mechanism to form a parallelogram and to constrain the movement of the tool drive 220 around a mechanical remote center of motion (RCM). The first segment may be referred as the setup arm because it may position and adjust the RCM in space relative to the mounting fixture, while the second segment may be referred to as the spherical arm because it is configured to move the surgical tool within a generally spherical workspace centered at the RCM. The RCM corresponds to the position on the patient's body wall through which a trocar or canular 221 can be inserted and surgical instrument 250 or a camera may enter.

Robotic arm 122 may be mounted to a surgical table, or a cart that may be separate from or removably coupleable to the surgical table, among other mounting fixtures. In a table-mount surgical robotic system, one or more arms can be mounted to an existing surgical table. A surgical table usually includes a tabletop, a support (or pedestal), and a base. The tabletop has a top surface on which a patient can be disposed. The support can provide for movement of the tabletop in several degrees of freedom. The robotic arms may be coupled to the support and/or the tabletop of the surgical table through a mounting interface. The mounting interface is often designed in such a way that the arms move together with the table when tabletop changes its pose. For example, when the tabletop translates along its longitude axis and/or tilts along its longitude or latitude axes, the arms move along while maintaining their respective relative positions and orientations to the tabletop. Therefore, no extra measures for patient's safety are needed if the patient remains still on the tabletop during these table motions. This is one of the most important benefits of table-mounted surgical robotic system.

### Coordinated Table Motion

In some implementations, remote center of motion (RCM) of the robotic arm refers to the point in space about which the spherical arm pivots the instrument and trocar as described above. Trocar markings allow users to align the RCM with the incision port on the patient during arm docking and port adjustment. Once the arm is docked to the trocar, the position of the RCM relative to the base of the arm is designed to remain constant. This helps minimize the amount of force exerted on the patient's body wall at the incision port as the trocar pivots during teleoperation. In a table-mounted surgical robotic system, the arms can be mounted to the patient table such that all the arms move together with the table. For instance, when the tabletop translates along its vertical and longitude axes and/or tilts along its longitude and latitude axes, the arms move along while maintaining their respective relative positions and orientations to the tabletop. Therefore, RCM remains relatively static and no extra measures for patient's safety are needed if the patient remains still on the tabletop during these table motions.

Coordinated table motion (also known as intraoperative table motion), in a table-mounted surgical robotic system in which the arms are mounted to the table, refers to this design in which bedside arms moving in coordination with the tabletop and patient so that arms do not need to be undocked from trocars when a patient is repositioned during a procedure. The table motions supported by the coordinated table motion include tabletop height, tilt, and Trendelenburg adjustment. While any of these table motions is occurring, the remote center position relative to the table (base of each arm) remains constant. Coordinated table motion can occur either during teleoperation or outside teleoperation when at least one arm is docked.

Note that coordinated table motion is analogous to but differs from integrated table motion for a cart-mounted surgical robot, in which arms are not coupled to the table and the system does not have exact knowledge of where the remote center is relative to the table. Robotic arms in such a system may enter a “floating state” during integrated table motion, where an external force is required to move the remote center of each arm so that it approximately follows table motion. In other words, the patient body wall is responsible for moving the remote center of each arm, via the interaction between the incision port and the trocar. Therefore, the force required to move the remote center for a cart mount surgical robot is often designed to be low (e.g., at approximately 2N), which reduces the likelihood of port trauma caused by the patient body wall pulling on the trocars. During integrated table motion, body wall forces are unlikely to increase significantly since the remote center will move easily to alleviate the force in response to any increased load.

On the other hand, the patient body wall plays no role in relocating the remote center in a table-mounted surgical robot during coordinated table motion. Arms are not required to be compliant to external force to enable table motion. In this case, the effective stiffness of the arms during coordinated table motion can be higher than cart-mounted arms, which could lead to a potential issue of excessive loading at the incision port, as the orientation of the patient changes with respect to gravity, and the force applied to the trocar by the body wall increases.

In intraoperative settings, a patient may experience excessive body wall forces during a procedure when either the patient is displaced or re-oriented in anyway against a stationary trocar, or the trocar is displaced against a stationary patient. For example, in a table-mounted surgical robot, table motions may cause the patient to be re-oriented. For a system with robotic manipulators mounted to a mobile cart separate from the surgical table, cart displacement can cause the same effect.

Potential excessive force at the incision port can be mitigated by a compliant RCM. In some implementations, the remote center is allowed to lower the effective stiffness to behave like a spring mechanism during coordinated table motion. Once enabled, the arm can sense the external force exerted by the body wall at the incision port and drift (or move) relative to the tabletop to be compliant with the external force before the system triggers a fault and stops table motion. The drift herein refers to the displacement of the RCM relative to the tabletop from its intended position, which can result in reduced external load being applied to the trocar or the arm. Certain compliance requirements can be defined, such as max drift distance and/or force threshold, based on safety regulation and common practices. For example, a surgical robot may allow a 20 mm RCM drift and/or a force threshold of 60N at the body wall during the coordinated table motion.

In conventional surgical robotic systems, before table movement occurs, a surgeon (or operator) may remove the surgical tool (and/or trocar) from an incision port of a patient and may move (or remove) the surgical arm before the table is adjusted. Once adjusted, the surgeon may then re-insert the surgical tool into the incision port to proceed with a surgical procedure. This may result in increased complexity and duration of a surgical procedure. The present disclosure, however, provides a method and system for enabling compliance of RCM due to external forces exerted by a body wall of an incision port of a patient caused by table movement. As a result, a surgical tool may remain inserted into the incision port while table movement occurs. In addition, the present disclosure enables end effector control (or manipulation), while table movement occurs since the RCM remains compliant.

### RCM Compliance

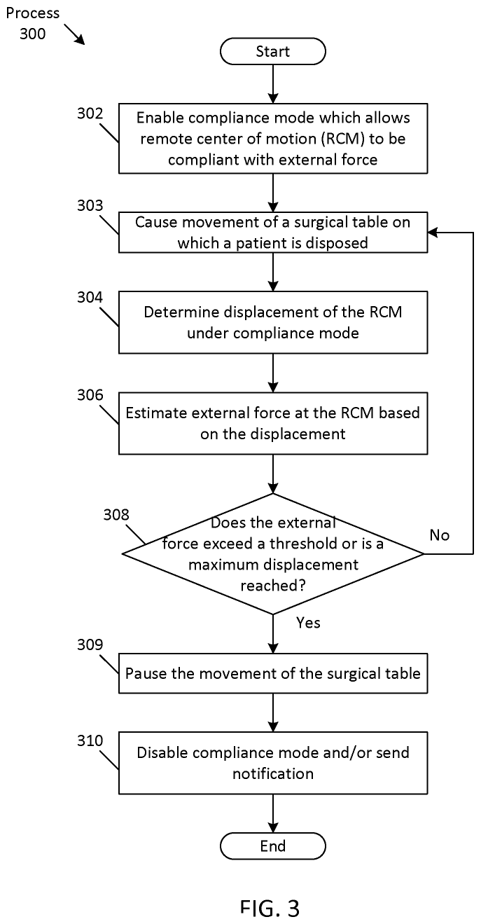

FIG. 3 is a flowchart of process 300 illustrating a method for detecting and mitigating excessive body wall force intraoperatively, in accordance with aspects of the subject technology. The method relies on force estimation of the contact force/torque applied on the trocar to ensure patient safety. Note that even though the method is designed for coordinated table motion, it can be applied to any surgical robotic system regardless of the mounting structure. As long as joints responsible for RCM are controlled to be compliant, maximum excessive force at RCM/body wall can be constrained by limiting the maximum displacement of RCM and safety measures can be taken accordingly.

In step 302, the system enables the compliance mode which allows RCM to be compliant with external force. This step may include operations, such as applying a spring mass damper model to RCM to change its stiffness and be compliant. One implementation to achieve this is to turn on spring damper at each joint of the robotic arm responsible for RCM. For example, in robotic arm 122 described in FIG. 2, joints 211-215 alone can determine the RCM and each takes part in enabling the RCM compliance. For a robotic arm with a mechanical RCM design and enough degrees of freedom, such as arm 122, joints responsible for RCM may be locked with a brake once the arm is docked to trocar. Therefore, step 302 may also include operations, such as releasing brakes and turning on gravity compensation at those joints, so that the arm would not collapse while allowing the RCM to drift.

In step 303, the system causes movement of a surgical table on which a patient is disposed. In particular, the patient may include a trocar within an incision port on the patient, where the RCM of the robotic arm may be aligned. The system may receive a command from a user (e.g., operating the user console 110) to perform one or more movements (or table motions), such as a rotation and/or translation. In one embodiment, the system may enable the compliance mode responsive to receiving the command to move the surgical table. In which case, once a command is received, the system may enable compliance mode and then cause (or being) the desired motion. In another embodiment, compliance mode may be enabled once the surgical table begins to move.

In step 304, the system determines displacement of RCM within the compliance mode. For example, the displacement value can be determined using encoders inside each joint of the arm responsible for RCM since one or more of those joints may have drifted to cause the RCM to drift in response to the force at body wall. In which case, the system may determine the RCM displacement based on one or more displacements sensed (or measured) at one or more joints of the RCM. As described herein, this RCM draft may be the result of the body wall pushing up against at least a portion of the docked trocar that is coupled to the robotic arm.

In step 306 the system estimates the force exerted by body wall based on the displacement determined in step 304. Specifically, this force may be caused by a patient re-orientation (e.g., due to table movement on which the patient is disposed) relative to the current RCM. In which case, the external force may be exerted by the body wall as the movement of the surgical table occurs. For example, as the table tilts, the body wall may push up against a trocar. As the table continues to move, the force exerted upon the trocar may increase. In compliance mode, force at the RCM can be inferred through RCM displacement. Since joints responsible for RCM are controlled to behave like a spring mechanism, a direct mathematic relationship exists between the force and displacement at each joint. Alternatively, force/torque at each joint can also be measured directly by torque sensors present at the joints. The contact force/torque with the patient body wall at the trocar can then be estimated based on joint force estimated (from joint displacements) or measurements (from torque/force sensor placed inside each joint) and the kinematic model of the robotic arm. In one embodiment, the external force is estimated based on a desired RCM stiffness and the determined RCM displacement. For example, the external force may be substantially proportional to the RCM displacement by a factor of the desired RCM stiffness. In one embodiment, the factor of the desired RCM stiffness may be predetermined. More about the desired RCM stiffness is described herein. In which case, the system 100 may be configured to estimate the external force at the RCM point using displacement determined from encoder readings (and/or kinematic model), without the need of an external sensor, such as a force sensor. In another embodiment, the system 100 may determine force based on sensor data.

Next, the displacement determined in step 304 may be compared to the max allowed displacement threshold and/or the external force estimated in step 306 may be compared with a force threshold in step 308. In one embodiment, the threshold for the external force may be a safety force threshold set for (e.g., predefined for) the compliance mode. In one embodiment, the system 100 may determine whether one or both criteria exceed their corresponding thresholds. For example, the system may determine whether the RCM displacement exceeds a maximum threshold, and if so, may proceed through process 300, but if not, the system may return to step 303 to continue the movement of the surgical table. In which case, at least some operations of process 300, such as those of step 306, may be optionally performed. In another embodiment, the system 100 may proceed when both thresholds are exceeded.

Returning to step 308, if it is determined that external force exceeds the force threshold and/or the maximum displacement has been reached, the system 100 may pause the movement of the surgical table at step 309. In particular, the system 100 may pause any movement such as rotations and/or translations that may be occurring as a result to a user command, for example. In step 310, the system 100 may disable the compliance mode and/or may notify the system user about the situation in step 310. In one embodiment, the system may disable compliance mode by preventing (or reducing) movement of at least the joints of the robotic arm that make up the RCM joints in response to any applied external forces. For instance, the system 100 may pause or prevent additional movement of at least the RCM joints of the robotic arm in order to keep the arm static. In some embodiments, the notification may be sent by displaying the (e.g., as a pop-up) notification on the user console 110, to alert the user. Specifically, the notification may alert the user to pause the teleoperation in order for the user to determine safety measures, such as adjusting the RCM through setup arms to fit with the re-oriented patient. In one embodiment, the notification may include a warning for a user of the surgical robotic system to stop all surgical table motions.

In one embodiment, the operations of step 309 may be in response to user action or may be automatically (e.g., without user intervention) performed by the system 100. For example, in response to determining that the external force exceeded the force threshold or that the maximum displacement has been reached, the system 100 may disable the compliance mode and send the notification to the user. Once the user receives the notification (e.g., a pop-up notification on a display of the system), the user may provide a user command to the system 100 to pause the movement of the surgical table in order to avoid any additional external force to be applied to the robotic arm. For example, the command may be the user releasing a clutch mechanism, which when activated allows the table to be moved. In another embodiment, the command may be a UI item displayed on the user screen.

Thus, the process 300 allows a user (e.g., surgeon) to effectively and efficiently re-orient the surgical table and/or the patient without needing to adjust the surgical robotic components and continue the teleoperation at the new pose. In particular, the operations of the system 100 allow the surgeon to adjust a surgical table without having to remove (undock) the robotic components, since such components remain complaint during a table movement.

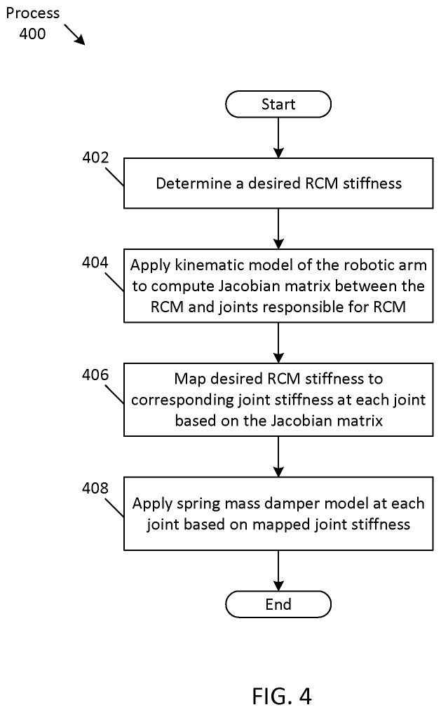

FIG. 4 is a is a flowchart illustrating an example design of compliance at a RCM for detecting and mitigating excessive body wall force intraoperatively, in accordance with aspects of the subject technology. For instance, at least some of the operations of process 400 may be performed in process 300, such as at step 302. In one embodiment, at least some of these operations may be performed in response to movement of the surgical table in order for the system to maintain a unified (constant) RCM point. As a result, once compliance mode is enabled and movement occurs, the system may dynamically perform at least some of the operations of process 400 to keep the RCM constant. Note that the example design can be applied to any robotic arm regardless of the RCM mechanism.

In step 402, the system determines a desired RCM stiffness. In one embodiment, the desired RCM stiffness may be based on the safety requirements of the compliance mode. For instance, these requirements may include the force threshold and max displacement of the RCM, as described in step 308 of process 300. In step 404, the system 100 may apply a kinematic model of the robotic arm to compute a Jacobian matrix between the RCM and the joints of the robotic arm responsible for the RCM. In step 406, the system 100 may map or translate the desired RCM stiffness to corresponding joint stiffness at each joint responsible for RCM based on the Jacobian matrix, which represents the kinematic relationship between different parts of the robotic arm. In particular, the system 100 may determine one or more joint stiffnesses for one or more joints that may be necessary in order to accomplish the desired RCM stiffness (in response to an applied external force upon the robotic arm, as described herein).

For example, in FIG. 2, assume joints 211, 212, and 213 are used for RCM admittance control, joints 214 and 215 are braked and RCM point has isotropic stiffness, i.e., K_{RCM} is a 3×3 stiffness matrix at the RCM point (e.g., due to having three joints used for RCM admittance control):

$${K}_{RCM} = [ \begin{matrix}{k}_{rcm} & 0. & 0. \\ 0. & {k}_{rcm} & 0. \\ 0. & 0. & {k}_{rcm}\end{matrix} ]$$

where k_{rcm}=Fₘₐₓ/dₘₐₓ. In this case, the desired RCM stiffness matrix may be determined, where Fₘₐₓ may be the maximum allowable contact force at the RCM point, and dₘₐₓ may be the maximum allowable displacement of the RCM point. Assuming a Jacobian of the RCM link position J_{RCM} is also a 3×3 matrix, the stiffness of joints 211-213 can be mapped from the RCM by:

$${K}_{J 1 - J 3} = {J}_{RCM}^{T} * {K}_{RCM} * {J}_{RCM} ,$$

where J₁-J₃ represents joints 211-213, respectively. The spatial Jacobian matrix for the RCM point can be calculated based on the kinematic model of the setup arm. This spatial Jacobian links the plurality of differential of joint displacements to the differential of the RCM point spatial linear displacement.

$${[ dx , dy , dz ]}^{T} = J * {[ {d}_{{θ}_{1}} , {d}_{{θ}_{2}} , {d}_{{θ}_{3}} ]}^{T}$$

In one embodiment, the matrix on the left may represent the linear displacement of the RCM point due to external force, while the matrix on right may represent the joint displacements (e.g., of each the RCM joints, such as 211-213). The transpose of the spatial Jacobian may also link the force applied to RCM point to the plurality of joint torques:

$${[ {τ}_{1} , {τ}_{2} , {τ}_{3} ]}^{T} = {J}^{T} * {[ {F}_{x} , {F}_{y} , {F}_{z} ]}^{T}$$

since the joint admittance controller may be controlling each joint to behave as a rotational spring. In one embodiment, the matrix on the left may represent torques on each joint, while the matrix on the right may represent the forces applied to the RCM point. Given that each of the plurality of joints is controlled as a spring mass damper system, each of the joints may have its own stiffness based on each of the joint's torque:

$${τ}_{i} = {k}_{i} * {d}_{{θ}_{i}}$$

where τᵢ may be joint torque, kᵢ may be joint stiffness, and d_{θ}_{ᵢ} may represent one or more displacements of a corresponding joint. Based on the Hooke's law for rotational springs at joints:

$${d}_{{θ}_{i}} = \frac{{τ}_{i}}{{k}_{i}} \to {[ {d}_{{θ}_{1}} , {d}_{{θ}_{2}} , {d}_{{θ}_{3}} ]}^{T} = {K}_{joint}^{- 1} * {[ {τ}_{1} , {τ}_{2} , {τ}_{3} ]}^{T}$$

Therefore, based on the three equations above:

$${[ dx , dy , dz ]}^{T} = J * {[ {d}_{{θ}_{1}} , {d}_{{θ}_{2}} , {d}_{{θ}_{3}} ]}^{T} = J * {K}_{Joint}^{- 1} * {[ {τ}_{1} , {τ}_{2} , {τ}_{3} ]}^{T} = J * {K}_{Joint}^{- 1} * {J}^{T} * {[ {F}_{x} , {F}_{y} , {F}_{z} ]}^{T}$$

To make the RCM act like a spring mass damper system with stiffness of K_{RCM}:

$${[ dx , dy , dz ]}^{T} = {K}_{RCM}^{- 1} * {[ {F}_{x} , {F}_{y} , {F}_{z} ]}^{T}$$

In one embodiment, since this may hold for any spatial force F, the compliance matrices may be equal to:

$${K}_{RCM}^{- 1} = J * {K}_{Joint}^{- 1} * {J}^{T}$$

With all these together, we have the mapping between the joint stiffness of the plurality of joints (e.g., under admittance control) and the desired RCM stiffness:

$${K}_{joint} = {J}^{T} * {K}_{RCM} * J$$

In one embodiment, K_{RCM} may ideally be a diagonal matrix with identical elements, which may be used to achieve isotropic RCM stiffness. In another embodiment, K_{RCM} may be an arbitrary matrix, such as a non-diagonal matrix. To achieve isotropic RCM stiffness, the system may be configured to use optimization (e.g., solving a linear quadratic programming problem) to determine an optimal joint stiffness. In one embodiment, the optimization may be formulated to find a positive definite diagonal joint stiffness matrix, Kⱼₒᵢₙₜ such that the calculated RCM stiffness matrix K_{RCM} is the closest to the desired isotropic stiffness matrix at the RCM point, Kiso, i.e., the Frobenius norm of the difference between these two matrixes may be minimized.

$${min}_{{k}_{joint}} {‖ {K}_{RCM}^{- 1} - {K}_{iso}^{- 1} ‖}_{F} \\ {K}_{joint} = arg {min}_{{k}_{joint}} {‖ ( J * {K}_{joint}^{- 1} * {J}^{T} ) - {K}_{iso}^{- 1} ‖}_{F}$$

In step 408 the system 100 applies a spring mass damper model at each joint based on the mapped joint stiffness, so that each joint can be compliant to achieve RCM compliance. In particular, the application of the damper model may enable each joint controller to reach (or apply) a stiffness required to achieve movement based on the applied external force to maintain a unified RCM point. As described herein, the system 100 may determine a displacement for each of the joints based on the movement of the robotic arm due to the mapped joint stiffness to determine whether RCM compliance should be disabled. For example, the spring mass damper can be implemented based on torque sensing in position control modes using an admittance controller. In another embodiment, the joint-level spring mass damper model may be implemented using position sensing coupled with torque control using an impedance controller. In one embodiment, the application of the spring mass damper model may compute each joint stiffness (associated with RCM compliance) according to the robotic arm kinematics model.

In one embodiment, the process 400 may allow the system 100 to (attempt to) maintain a unified stiffness at the RCM point by adjusting one or more joint stiffnesses based on the (changing) configuration of the arm (e.g., due to body wall force applied to the arm). In particular, if the (one or more joints) robotic arm (responsible for maintaining the RCM) move from one configuration to another configuration, the stiffness of the joint(s) may change, but at least some of the operations of process 400 may enable the RCM point to reach (approximately) the same stiffness between the configurations.

As described herein, the system 100 may be configured to achieve compliance by controlling the position of one or more joints of the robotic arm. In order to be compliant to an external force applied to a portion of the arm, the system may be configured to calculate and perform movements of one or more of the joints. To remain compliant, the system may calculate a desired joint position through a position command and/or through a torque command.

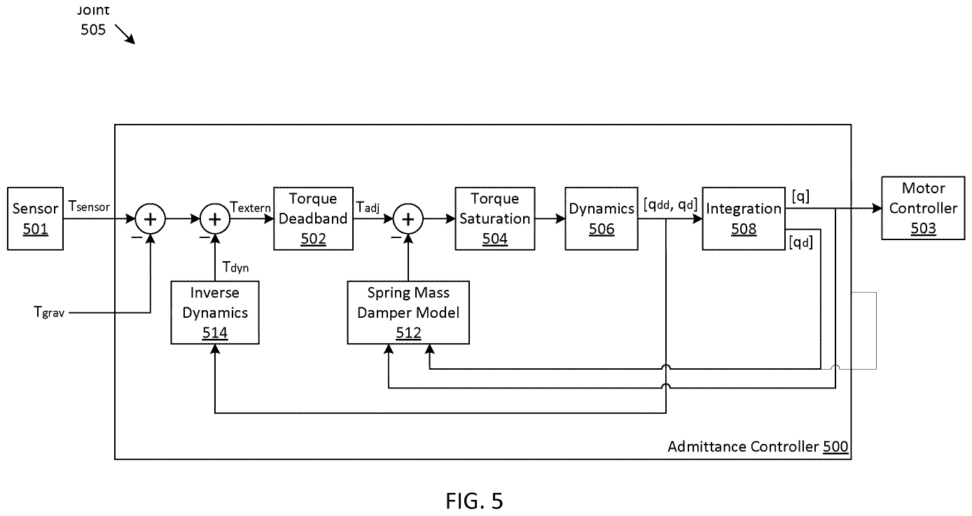

FIG. 5 illustrates a joint 505 as a block diagram that includes one example design of such a joint-level controller for RCM compliance, in accordance with aspects of the subject technology. As shown in FIG. 5, the joint includes a sensor 501, an admittance controller 500, and a motor controller 503. The sensor 501 may be a torque sensor arranged to measure torque of the joint when a force (e.g., rotational force) is applied to the joint. In one aspect, the sensor may measure torque applied to the joint (e.g., by one or more links of the robotic arm) in response to the external force applied to the arm, as described herein. In one embodiment, the sensor 501 may produce a torque output, Tₛₑₙₛₒᵣ, which may be a measured/sensed torque at output of the joint 505. In one embodiment, Tₛₑₙₛₒᵣ may include torques for inherent dynamics and gravity compensation, as well as external forces. The motor controller 503 may be configured to receive one or more (e.g., position and/or torque) commands and may control one or more motors (not shown) of the joint 505 in order to perform one or more adjustments (e.g., rotational and/or translational) of the joint 505.

The joint 505 also includes an admittance controller 500 that may be configured to generate a position and/or velocity command (as output) based on current torque values (input) of the joint. In one embodiment, the admittance controller may be a part of the joint 505 in order to control movement of the joint, as shown. Although illustrated as being separate controllers, both the at least some of the operations of the admittance controller 500 may be performed by the motor controller 503. In another embodiment, the admittance controller may be configured to generate position and/or velocity commands for one or more joints. In which case, the system 100 may include an admittance controller that may be configured to receive sensor data from one or more sensors of one or more joints and may be configured to produce one or more position and/or velocity commands for those one or more joints in order to provide RCM compliance, as described herein.

The admittance controller 500 includes one or more operational blocks, such as a torque deadband 502, a torque saturation 504, dynamics 506, an integration 508 on a forward loop, and a spring mass damper model 512 and an inverse dynamics 514, both on feedback loops. In one embodiment, the controller may include less or more blocks, such as omitting the torque deadband 502. In another embodiment, the dynamics 506 may be a part of or communicatively coupled with one or more motors and/or one or more sensors of the joint 505. The admittance controller may be configured to receive torque measurement data, Tₛₑₙₛₒᵣ, from the sensor 501 and may receive a gravity compensation torque, T_{grav}, which may be torque on the joint due to gravity. In one embodiment, T_{grav} may be a predefined torque value. The admittance controller may subtract T_{grav} from the Tₛₑₙₛₒᵣ.

The controller 500 may be configured to subtract T_{grav} from Tₛₑₙₛₒᵣ to remove the effect of gravity on the joint. The controller 500 may remove the torque output of the inverse dynamics 514, which is configured to calculate torque based on the joint velocity and acceleration to produce an external torque, Tₑₓₜₑᵣₙ, from external forces on the robot arm (e.g., not from gravity or dynamics). In particular, the inverse dynamics 514 may be configured to receive acceleration, q_{dd}, and velocity q_{d}, and may be configured to produce joint torque, T_{dyn}, due to joint acceleration. In particular, the inverse dynamics may be configured to calculate T_{dyn} based on at least one of position, q, velocity, q_{d}, and acceleration, q_{dd}. The T_{dyn} produced by the inverse dynamics 514 may be the torque on the joint due to inertia effect of the joint. In one embodiment, the inverse dynamics 514 may determine the joint torque based on a configuration of the entire robotic arm (e.g., positions of one or more joints of the arm), which may be determined based on encoder data and/or arm kinematics. In some embodiments, T_{dyn} may include torque due to at least one of gravity, acceleration, which may come from inverse kinematics via a kinematic mode, and link center of gravity. In one embodiment, the admittance controller 500 may be configured to determine Tₑₓₜₑᵣₙ as the difference between Tₛₑₙₛₒᵣ (which may be compensated for T_{grav}) and T_{dyn}. In some embodiments, Tₑₓₜₑᵣₙ may be the external torque at each joint associated with RCM, which may be calculated by subtracting gravity torque and the dynamic torque from the inverse dynamics 514 from the corresponding joint output torque. As a result, the controller 500 may be configured to get the actual external torque at the joint.

The torque deadband 502 may be configured to determine whether the input torque, Tₑₓₜₑᵣₙ, is greater than a torque deadband value. In particular, the deadband may be configured to receive Tₑₓₜₑᵣₙ, which may include Tₛₑₙₛₒᵣ (compensated for T_{grav} and/or T_{dyn}), and determine whether the input torque is greater than a threshold. If not, the deadband may output a low (or minimum) torque, and as a result the admittance controller may not cause any position command (or change in position). Therefore, if the external torque sensed by the torque sensor is smaller than a threshold, the system may not adjust the joint 505. In one embodiment, this low torque may be (approximately) zero. If, however, the input torque is greater than the threshold, the deadband may output T_{adj}, which may be based on the input Tₑₓₜₑᵣₙ. Specifically, the torque deadband 502 may pass through Tₑₓₜₑᵣₙ and T_{adj}.

The torque saturation 504 may be configured to clamp the joint admittance control torque to a torque saturation value based the output of torque deadband 502, T_{adj}. If the torque value output from the torque deadband 502 is greater than a maximum allowable value (e.g. maximum threshold), the torque saturation 504 may cap the torque with a saturation value. In one aspect, the torque saturation 504 may receive a difference between T_{adj} and output of the spring mass damper model 512, which may be configured to determine joint torque.

The dynamics 506 produces (or solves for) the joint acceleration q_{dd} and velocity q_{d} based on the saturated joint admittance control torque output from the torque saturation 504. In one embodiment, the dynamics may be (coupled to) a controlled plant, such as a joint motor of the joint 505. In which case, the joint acceleration and velocity may be produced based on filtered sensor readings of one or mor sensors of the joint in response to (or based on) the saturated joint admittance control torque output of the torque saturation 504. The output from the dynamics 506 may be converted into a desired (angular) position q that the joint 505 is to be moved to by integration 508. The motor controller 503 may be configured to receive q as a position command for the joint 505, and use the input to adjust (or as a position command for) the joint. In one embodiment, the output of the integration may be used as position command for each joint.

In one embodiment, the output of the dynamics 506, i.e., joint acceleration q_{dd} and velocity q_{d}, may be fed back to inverse dynamics 514 to calculate the torque value (which is based on all joint positions of the robotic arm, as described herein) to be subtracted from the Tₛₑₙₛₒᵣ (which may be compensated for T_{grav}) to produce Tₑₓₜₑᵣₙ based on feedback from the integration 508. In another embodiment, the spring mass damper model 512 may determine the joint torque due to output from the integration 508, which may include q_{d} and q, the potential desired position. Specifically, the spring mass damper model 512 may determine the impedance model torque based on input joint value and velocity. This output of the impedance model may be a desired torque in order for the system to mimic the position of the joint according to the spring mass damper model, as described in process 400 of FIG. 4. In particular, the spring mass damper model 512 may be configured to receive the desired joint stiffness determined in process 400 of FIG. 4 in order to maintain the unified RCM point, and may determine the desired torque according to the desired joint stiffness with respect to q_{d} and q. In one embodiment, the desired torque may be output by the model responsive to input based on at least one of the desired joint stiffness, q_{d}, or q. In one embodiment, the model 512 may be configured to perform at least some of the operations of process 400 in FIG. 4 to determine a desired torque in response to the velocity and position with respect to one or more parameters, such as stiffness.

As described thus far, the spring mass damper may be implemented using the admittance controller 500 of FIG. 5. Alternatively, the spring mass damper may be implemented based on position sensing in torque control modes using an impedance controller. In this implementation, the impedance controller may generate a joint torque command (output) based on current joint angle/velocity (input) and desired joint angle/velocity (input).

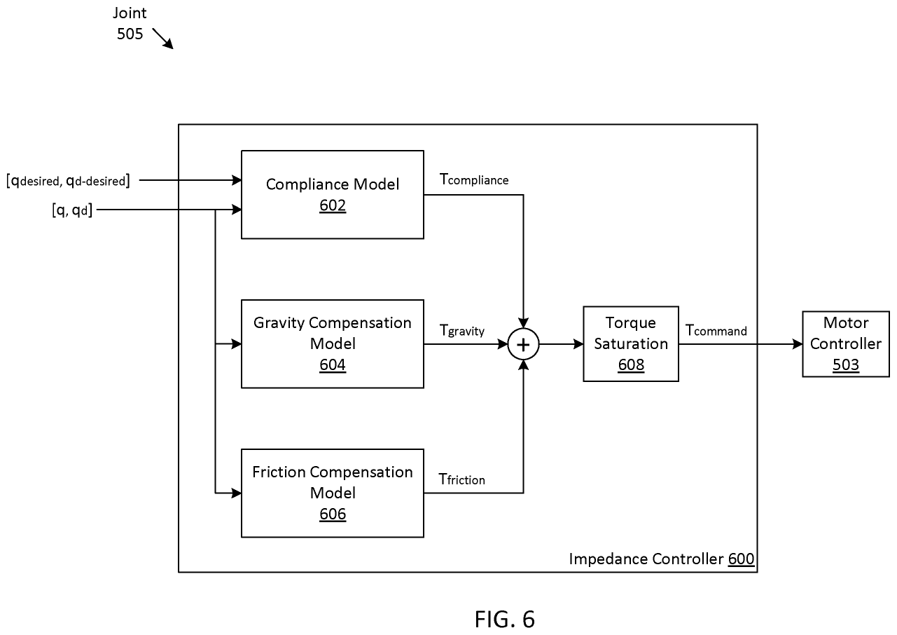

FIG. 6 illustrates the joint 505 as a block diagram illustrating another example design of such a joint-level controller for RCM compliance, in accordance with aspects of the subject technology. As shown, the joint has an impedance controller 600 which includes a compliance model 602, a gravity compensation model block 604, a friction compensation model block 606, and a torque saturation block 608.

The impedance controller 600 may be configured to receive a current joint angle, q, and current velocity, q_{d}, for the joint 505. In one embodiment, this input may be received from a sensor (or encoder) of the joint. The impedance controller may also receive a desired joint angle q_{desired} and a desired velocity, q_{d-desired}, which may be based on requirements of the compliance mode of the system 100.

The compliance model 602 may be configured to take takes input of q and q_{d}, as well q_{desired} and q_{d-desired}, to compute compliance torque, T_{compliance}. In one embodiment, the compliance model block may implant a mass-spring damper system in order to determine T_{compliance} to move the desired input based on the current joint input. In particular, the compliance model may use the mass-spring damper system to determine a desired joint stiffness, which may be used to determine the torque of the joint in order to remain in compliance with the RCM point, as described herein in order to be compliant to external force(s).

The gravity compensation model block 604 and friction compensation model block 606 may calculate the gravity compensation torque, T_{gravity}, and the friction compensation torque, T_{friction}, respectively, based on current joint angle q and velocity q_{d}. In particular, the gravity compensation model compensates for the gravity based on the current position (and/or velocity) of the robotic arm. The friction compensation model block produces the friction compensation torque in order to overcome the effects of friction upon the (moving parts of the) joint. In one embodiment, both of these models may be predefined models based on the structure (specification) of the joint.

Next, sum of the torque values (T_{compliance}+T_{gravity}+T_{friction}) may be passed through torque saturation block 608 to generate the joint torque command, T_{command}, and make sure the torque command is well capped. The joint torque command can then be sent to motor controller 503 to drive the motor accordingly.

As described thus far, the system 100 may be configured to determine displacement of the RCM under compliance mode using data, such as information from encoders of one or more joints that make up (or create) the RCM. In another embodiment, the system 100 may be configured to determine displacement through one or more external sensors, such as image sensors (or cameras). In particular, the system may be configured to track the body wall movement of the patient captured by one or more cameras, and determine displacement of the RCM. In another embodiment, the system may track at least a portion of the robotic arm, such as the trocar, to determine whether movement occurs. If the RCM moves beyond a threshold, the system may disable compliance and/or pause the movement of the robotic table, as described herein.

In one embodiment, an RCM displacement may be determined by the system 100 based on displacements sensed at the RCM joints. Returning to the previous example, when the joints responsible are joints 211, 212, and 213 in FIG. 2, the system may determine the RCM displacement based on movement of each of these joints. In some embodiments, the external force on the RCM may be caused by a patient re-orientation relative to the current RCM. In another embodiment, the external force on the RCM may be caused by RCM displacement.

The foregoing description, for purposes of explanation, used specific nomenclature to provide a thorough understanding of the disclosure. However, it will be apparent to one skilled in the art that specific details are not required in order to practice the disclosure. Thus, the foregoing descriptions of specific embodiments of the disclosure are presented for purposes of illustration and description. They are not intended to be exhaustive or to limit the disclosure to the precise forms disclosed; obviously, many modifications and variations are possible in view of the above teachings. The embodiments were chosen and described in order to best explain the principles of the disclosure and its practical applications, they thereby enable others skilled in the art to best utilize the disclosure and various embodiments with various modifications as are suited to the particular use contemplated. It is intended that the following claims and their equivalents define the scope of the disclosure.

The methods, devices, processing, and logic described above may be implemented in many different ways and in many different combinations of hardware and software. The controllers and estimators may comprise electronic circuitry. For example, all or parts of the implementations may be circuitry that includes an instruction processor, such as a Central Processing Unit (CPU), microcontroller, or a microprocessor; an Application Specific Integrated Circuit (ASIC), Programmable Logic Device (PLD), or Field Programmable Gate Array (FPGA); or circuitry that includes discrete logic or other circuit components, including analog circuit components, digital circuit components or both; or any combination thereof. The circuitry may include discrete interconnected hardware components and/or may be combined on a single integrated circuit die, distributed among multiple integrated circuit dies, or implemented in a Multiple Chip Module (MCM) of multiple integrated circuit dies in a common package, as examples.

The circuitry may further include or access instructions for execution by the circuitry. The instructions may be stored in a tangible storage medium that is other than a transitory signal, such as a flash memory, a Random Access Memory (RAM), a Read Only Memory (ROM), an Erasable Programmable Read Only Memory (EPROM); or on a magnetic or optical disc, such as a Compact Disc Read Only Memory (CDROM), Hard Disk Drive (HDD), or other magnetic or optical disk; or in or on another machine-readable medium. A product, such as a computer program product, may include a storage medium and instructions stored in or on the medium, and the instructions when executed by the circuitry in a device may cause the device to implement any of the processing described above or illustrated in the drawings.

The implementations may be distributed as circuitry among multiple system components, such as among multiple processors and memories, optionally including multiple distributed processing systems. Parameters, databases, and other data structures may be separately stored and managed, may be incorporated into a single memory or database, may be logically and physically organized in many different ways, and may be implemented in many different ways, including as data structures such as linked lists, hash tables, arrays, records, objects, or implicit storage mechanisms. Programs may be parts (e.g., subroutines) of a single program, separate programs, distributed across several memories and processors, or implemented in many different ways, such as in a library, such as a shared library (e.g., a Dynamic Link Library (DLL)). The DLL, for example, may store instructions that perform any of the processing described above or illustrated in the drawings, when executed by the circuitry.

Also, the various controllers discussed herein can take the form of processing circuitry, a microprocessor or processor, and a computer-readable medium that stores computer-readable program code (e.g., firmware) executable by the (micro) processor, logic gates, switches, an application specific integrated circuit (ASIC), a programmable logic controller, and an embedded microcontroller, for example. The controller can be configured with hardware and/or firmware to perform the various functions described below and shown in the flow diagrams. Also, some of the components shown as being internal to the controller can also be stored external to the controller, and other components can be used.

Some embodiments may perform variations to at least some of the processes described herein. For example, the specific operations of at least some of the processes may not be performed in the exact order shown and described. The specific operations may not be performed in one continuous series of operations and different specific operations may be performed in different embodiments. For example, at least some operations may be optional operations that may not be performed while (or each time) a respective process is performed. In one embodiment, at least some of the operations described herein (e.g., performed in one or more processes described herein) may be performed automatically (e.g., without user interference). For example, at least some operations may be performed at any stage during a surgical procedure. In some embodiments, at least some of the operations described herein may be performed (e.g., continuously) in real-time (e.g., while a robotic surgical system is in use).

As previously explained, an embodiment of the disclosure may be a non-transitory machine-readable medium (such as microelectronic memory) having stored thereon instructions, which program one or more data processing components (generically referred to here as a “processor”) to (automatically) detect and mitigate excessive body wall force upon at least a portion of a robotic arm during an intraoperative procedure, as described herein. In other embodiments, some of these operations might be performed by specific hardware components that contain hardwired logic. Those operations might alternatively be performed by any combination of programmed data processing components and fixed hardwired circuit components.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

While certain embodiments have been described and shown in the accompanying drawings, it is to be understood that such embodiments are merely illustrative of and not restrictive on the broad disclosure, and that the disclosure is not limited to the specific constructions and arrangements shown and described, since various other modifications may occur to those of ordinary skill in the art. The description is thus to be regarded as illustrative instead of limiting.

In some embodiments, this disclosure may include the language, for example, “at least one of [element A] and [element B].” This language may refer to one or more of the elements. For example, “at least one of A and B” may refer to “A,” “B,” or “A and B.” Specifically, “at least one of A and B” may refer to “at least one of A and at least one of B,” or “at least of either A or B.” In some embodiments, this disclosure may include the language, for example, “[element A], [element B], and/or [element C].” This language may refer to either of the elements or any combination thereof. For instance, “A, B, and/or C” may refer to “A,” “B,” “C,” “A and B,” “A and C,” “B and C,” or “A, B, and C.”

1. A method comprising:
enabling a compliance mode in controlling a robotic arm of a surgical robot to allow a remote center of motion (RCM) of the robotic arm to be compliant with an external force exerted;
determining an RCM displacement under the compliance mode in response to the external force; and
responsive to determining that the RCM displacement exceeds a maximum threshold, disabling the compliance mode and sending a notification.

2. The method of claim 1, wherein the robotic arm is mounted on a surgical table on which a patient is disposed, wherein the method further comprises causing movement of the surgical table, wherein the external force is exerted on the RCM by an incision port on body wall of the patient as the movement of the surgical table occurs.

3. The method of claim 2 further comprising pausing the movement of the surgical table responsive to the determining that the RCM displacement exceeds the maximum threshold or to receiving a user command after the notification is sent.

4. The method of claim 1, wherein the robotic arm comprises a plurality of joints, wherein enabling the compliance mode comprises:
determining a desired RCM stiffness;
applying a kinematic model of the robotic arm to compute a Jacobian matrix between the RCM and the plurality of joints;
mapping the desired RCM stiffness to corresponding joint stiffness at each of the plurality of joints based on the Jacobian matrix; and
applying a spring mass damper model at each of the plurality of joints based on the mapped joint stiffness.

5. The method of claim 4 further comprising estimating the external force based on the desired RCM stiffness and the determined RCM displacement, wherein the compliance mode is disabled, and the notification is sent responsive to determining that the external force exceeds a force threshold.

6. The method of claim 5, wherein the external force is proportional to the RCM displacement by a factor of the desired RCM stiffness.

7. The method of claim 4, the spring mass damper model is applied using either 1) an admittance control that generates a position output based on torque input at each of the plurality of joints or 2) impedance control that generates a joint torque output based on current joint angle and desired joint angle at each of the plurality of joints.

8. The method of claim 1, wherein a patient is disposed on a surgical table and the external force is caused by a portion of the patient pushing up against a surgical instrument coupled to the robotic arm due to surgical table motion, wherein the notification comprises a warning for a user of the surgical robot to stop all surgical table motions.

9. A surgical robotic system, comprising:
a robotic arm having a plurality of joints and a remote center of motion (RCM) aligned with an incision port on a body wall of a patient;
at least one processor; and
memory having instructions stored therein which when executed by the at least one processor causes the surgical robotic system to:
enable a compliance mode in controlling the robotic arm, the compliance mode allows the RCM to be compliant with an external force exerted by the body wall around the incision port,
determine an RCM displacement under the compliance mode in response to the external force; and
responsive to determining that the RCM displacement has reached a maximum displacement of the RCM, disable the compliance mode and send a notification.

10. The surgical robotic system of claim 9, wherein the memory has further instructions to estimate the external force exerted by the body wall based on the determined RCM displacement, wherein the compliance mode is disabled, and the notification is sent responsive to determining that the external force exceeds a force threshold, and that the RCM displacement has reached the determined maximum displacement.

11. The surgical robotic system of claim 10, wherein the instructions to enable the compliance mode comprises instructions to:
determine a desired RCM stiffness based on the force threshold and the maximum displacement of the RCM;
apply a kinematic model of the robotic arm to compute a Jacobian matrix between the RCM and the plurality of joints;
map the desired RCM stiffness to corresponding joint stiffness at each of the plurality of joints based on the Jacobian matrix; and
apply a spring mass damper model at each of the plurality of joints based on the mapped joint stiffness.

12. The surgical robotic system of claim 11, wherein the external force is determined based on the desired RCM stiffness and the determined RCM displacement, wherein the external force is proportional to the RCM displacement by a factor of the desired RCM stiffness.

13. The surgical robotic system of claim 11, wherein the spring mass damper model is applied using either 1) an admittance control that generates a position output based on torque input at each of the plurality of joints or 2) impedance control that generates a joint torque output based on current joint angle and desired joint angle at each of the plurality of joints.

14. The surgical robotic system of claim 9, wherein the patient is disposed on a surgical table and the external force is caused by surgical table motion, wherein the notification comprises a warning for a user to stop all surgical table motion.

15. The surgical robotic system of claim 14, wherein the robotic arm is mounted on the surgical table.

16. The surgical robotic system of claim 14, wherein the robotic arm is mounted on a cart that is separate from the surgical table.

17. A non-transitory machine-readable medium storing instructions operable to cause one or more processors to perform operations including:
enabling a compliance mode in controlling a robotic arm of a surgical robot, wherein the robotic arm includes a remote center of motion (RCM) aligned with an incision port on a body wall of a patient, and the compliance mode allows the RCM to be compliant with an external force exerted by the body wall around the incision port;
determining an RCM displacement under the compliance mode in response to the external force;
estimating the external force based on the determined RCM displacement; and
responsive to determining that the external force exceeds a force threshold, or the RCM displacement reaches a maximum displacement, disabling the compliance mode and sending a notification.

18. The non-transitory machine-readable medium of claim 17, wherein the robotic arm comprises a plurality of joints, wherein enabling the compliance mode comprises:
determining a desired RCM stiffness based on the force threshold or the maximum displacement;
applying a kinematic model of the robotic arm to compute a Jacobian matrix between the RCM and the plurality of joints;
mapping the desired RCM stiffness to corresponding joint stiffness at each of the plurality of joints based on the Jacobian matrix; and
applying a spring mass damper model at each of the plurality of joints based on the mapped joint stiffness.

19. The non-transitory machine-readable medium of claim 18, wherein the external force is determined based on the desired RCM stiffness and the determined RCM displacement, wherein the external force is proportional to the RCM displacement by a factor of the desired RCM stiffness.

20. The non-transitory machine-readable medium of claim 17, wherein the external force on the RCM is caused by a patient re-orientation relative to the RCM.

21. The non-transitory machine-readable medium of claim 20, wherein the patient re-orientation is caused by motions of a surgical table, on which the patient is disposed.
